(19) United States
(12) Patent Application Publication
(10) Pub. No.: US 2009/0298918 A1
(43) Pub. Date: Dec. 3, 2009

(54) Alternative Splicing Isoform of Lox-I Protein Encoding Gene, and Uses Thereof

(75) Inventors: Giuseppe Novelli, Roma (IT); Ruggero Mango, Roma (IT); Francesco Romeo, Roma (IT); Renato Lauro, Roma (IT); Fabrizio Clementi, Roma (IT)

Correspondence Address: YOUNG & THOMPSON, 209 Madison Street, Suite 500, ALEXANDRIA, VA, 22314, US

(73) Assignee: UNIVERSITA' DEGLI STUDI ROMA TOR VERGATA, Roma (IT)

(21) Appl. No.: 11/922,877

(22) PCT Filed: Jun. 20, 2006
(86) PCT No.: PCT/IT2006/000470

§ 371 Date: Apr. 1, 2008

(30) Foreign Application Priority Data
Jun. 23, 2005 (IT) RM2005A000325

(51) Int. Cl. A61K 48/00; C12Q 1/68; A61P 9/00; C07H 21/04

(52) U.S. Cl. 514/44 R; 435/6; 536/24.3

(57) ABSTRACT

The invention relates to a new alternative splicing isoform of OLR1 gene encoding for the LOX-1 protein, uses and methods related to the treatment and to the prediction of the risk of cardiovascular diseases.

The present invention relates to a new isoform of alternative splicing of the OLR1 gene encoding for the LOX-1 protein, uses and methods related to the treatment and to the prediction of the risk of cardiovascular pathologies. More particularly, the present invention refers to methods for the prediction of the risk to develop cardiovascular pathologies, such as atherosclerosis, myocardial infarction, coronary artery disease, cerebral stroke, ischemia, acute and chronic peripheral vascular pathologies.

The atherosclerosis is a multifactorial disease with genetic component that represents the principal cause of mortality and morbidity in the industrialized countries. The main clinical manifestations of the atherosclerosis are represented by coronary artery disease (CAD), acute myocardial infarction (AMI), cerebral stroke and peripheral vascular disease. Recent studies have shown that atherogenesis is no longer an inevitable consequence of aging but instead is a chronic disease whose genesis both individual factors, mainly with a strong genetic component (hypertension, hypercholesterolemia, diabetes) and environmental (smoking, diet, stress) contribute to (Binder CJ, et al., Nat Med. 8; 1218-1226; Steinberg, D. Nat Med. 8; 1211-1217).

Recently several epidemiological studies highlighted newly defined non-traditional risk factors that are emerging as being equally important in atherosclerosis pathogenesis (Steinberg, D. Nat Med. 8; 1211-1217). Among these oxidized low-density lipoprotein (OxLDL) are considered to play a fundamental role in the entire process of atherogenesis. OxLDL elicits endothelial dysfunction, a key step in the initiation of atherosclerosis plaque, favouring generation of reactive oxygen species (free raduicals), inhibition of nitric oxide synthesis, and enhancement of monocytes adhesion to activated endothelial cells (Mehta J L and Them D. J Am Coll Cardiol. 39; 1429-1435; Chen M, et al., Pharmacol Ther. 95; 89-10).

In addition, OxLDL are involved in inducing smooth muscle cell migration and proliferation, and in foam cells formation, crucial events of atherogenesis (Chen M et al., Pharmacol Ther. 95; 89-10). Increased plasmatic levels of OxLDL were associated to plaque instability and to acute coronary syndromes (MI and unstable angina). In fact as shown, plasmatic OxLDL levels show a significant positive correlation with the severity of acute coronary syndromes and atherosclerotic lesions. Instable plaques contain significantly higher percentage of OxLDL-positive macrophages (Ehara S, et al., Circulation. 103; 1955-1960) and of apoptotic cells. According to this scenario, it has been shown that OxLDL are cytotoxic by inducing necrosis and apoptosis of different cells involved in the process of atherogenesis (smooth muscle cells, endothelial cells, and macrophages)(Chen J, et al., Circ Res. 94; 269-270; Hardwick S J, et al., J. Pathol. 179; 294-302; Sata M, et al., J Clin Invest. 102; 1682-1682; Lee T S and Lee Y C, Am J. Physiol. 280; 709-718). These processes have been proposed to lead to atherosclerotic plaque destabilization and subsequent rupture with acute atherothrombotic vascular occlusion and tissue infarction.

Most of the above mentioned effects are determined by the interaction of OxLDL with their major receptor, named LOX-1. LOX-1 receptor is encoded from the OLR1 gene. Such gene, mapped on the 12p12.3-13.2 chromosomal region, is constituted by 6 exons and 5 introns.

LOX-1 is a type-II membrane protein belonging to the C-type lectin family. LOX-1 consists of 4 domains: a short N-terminal cytoplasmic domain, a transmembrane region, a connecting neck, and a lectin-like domain at the C terminus whose integrity is critically required for its binding activity (Sawamura T, et to the., Nature. 386; 73-77; Shi X, et al., J Cell Sci. 114; 1273-1282). LOX-1 is expressed in endothelial cells, macrophages, smooth muscle cells and platelets. Furthermore, LOX-1 is present in atheroma derived cells and is overexpressed in humans and animal atherosclerotic lesions (Kataoka H, et al., Circulation. 99; 3110-3117).

On the basis of the above scientific evidences the authors of the present invention have reported a statistically significant association between polymorphisms in the OLR1 gene and myocardial infarction susceptibility (Mango R, et al., J Med Genet. 40; 933-936).

In the light of the above it is evident the need to have new rapid and not invasive methods for risk assessment of cardiovascular diseases, such as for instance, myocardial infarction, cerebrovascular insufficiency and peripheral vascular disease. It is also desirable to be able to have new therapeutic targets for the treatment of the aforementioned cardiovascular diseases for example through the inhibition of the formation, progression and destabilization of the atherosclerotic plaque.

The authors of the present invention have now identified and characterized a splicing isoform of the OLR1/LOX-1 gene, encoding for a protein named LOXIN that is shown to be protective against the risk to develop cardiovascular diseases such as myocardial infarction.

Through functional studies by real-time PCR, cloning and western blot analysis the authors have succeeded in identifying a new protein isoform of the LOX-1 receptor, named LOXIN, that is produced for alternative splicing of the OLR1 gene. Functional studies performed on the OLR1 gene transcript have shown how the expression of the two isoforms (OLR1/LOX-1 full length and LOXIN) is modulated by the polymorphisms previously identified by the same authors and located in the introns 4,5 and 3'UTR of the OLR1 gene. In particular, subjects carrying the risk haplotype for myocardial infarction at the polimorphisms located in the linkage disequilibrium block show a decreased expression of LOXIN and an increased expression of LOX-1 at mRNA and protein level.

Accordingly the authors have set also a rapid, economic and reliable method for the quantification of LOXIN and LOX-1 transcripts using Real-Time PCR (ABI 7000 device) starting from peripheral blood for the prediction of the risk of cardiovascular diseases, such as for instance, myocardial infarction, cerebrovascular insufficiency and peripheral vascular disease.

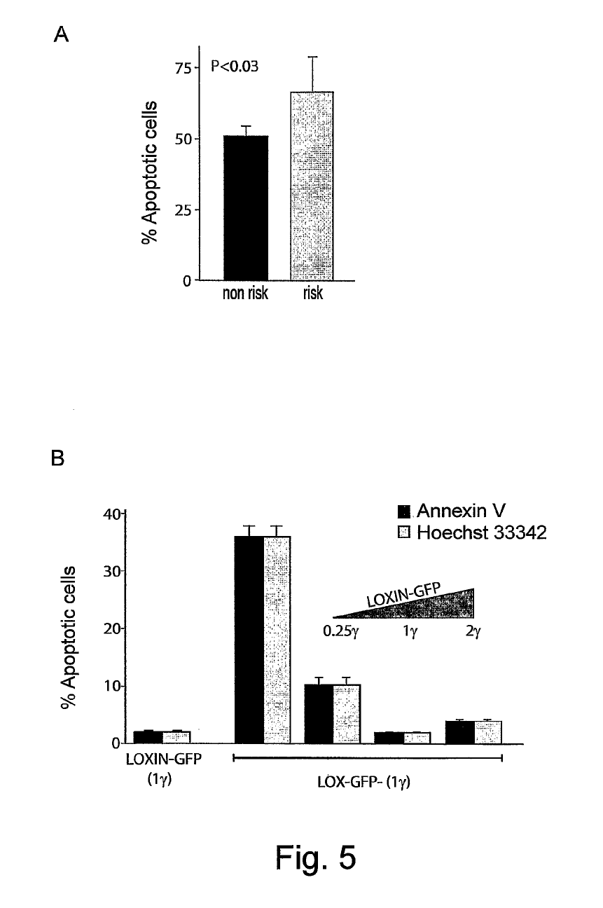

Besides as shown by flow cytometry and immunofluorescence experiments, the new identified LOXIN isoform is able to reduce OxLDL induced cytotoxicity reducing apoptosis degree by 40%. Since the apoptosis is a key step in atherosclerotic plaque destabilization, the experimental evidence of the reduction of the apoptotic index after addition of LOXIN in cell cultures treated with OxLDL or transfected with LOX-1, addresses LOXIN as possible molecular marker of plaque instability and molecular stabilizer of the atherosclerotic plaque as therapeutic implication. Such data are supported by in vivo experiments carried out on subjects with myocardial infarction that have shown how the macrophages of such patients, expressing a smaller quantity of LOXIN, are more susceptible to the apoptotic damage oxLDL induced (FIG. 5 panel A).

It is therefore an object of the present invention an alternative splicing isoform of the OLR1 gene, encoding for the oxidized low density lipoproteines receptor-1 (LOX-1), characterized in that exon 5 from the nucleotide 565 to the nucleotide 680 is excised in comparison to the corresponding native oligonucleotidic sequence of the human OLR1 transcript (NM 002543) or its complementary sequence.

In a preferred embodiment of the present invention the above mentioned alternative splicing isoform comprises the following oligonucleotidic sequence:

atgacttttg atgacctaaa gatccagact gtgaaggacc agcctgatga gaagtcaaat ggaaaaaaag ctaaaggtct tcagtttctt tactctccat ggtggtgcct ggctgctgcg actctagggg tcctttgcct gggattagta gtgaccatta tggtgctggg catgcaatta tcccaggtgt ctgacctcct aacacaagag caagcaaacc taactcacca gaaaaagaaa ctggagggac agatctcagc ccggcaacaa gcagaagaag cttcacagga gtcagaaaac gaactcaagg aaatgataga aacccttgct cggaagctga atgagaaatc caaagagcaa atggaacttc accaccagaa tctgaatctc caagaaacac tgaagagagt agcaaattgt tcagctcctt gtccgcaaga ctggatctgg catggagaaa actgttacct attttcctcg ggctcattta actgggaaaa cagccaagag aagtgcttgt ctttggatgc caagttgctg aaaattaata gcacagctga tctggacttc atccagcaag caatttccta ttccagtttt ccattctgga tggggctgtc tcggaggaac cccagctacc catggctctg ggaggacggt tctcctttga tgccccactt atttagagtc cgaggcgctg tctcccagac atacccttca ggtacctgtg catatataca acgaggagct gtttatgcgg aaaactgcat tttagctgcc ttcagtatat gtcagaagaa ggcaaaccta agagcacagt ga (SEQ ID NO:1) or its complementary sequence.

According to a further embodiment of the present invention the above defined alternative splicing isoform comprises an oligonucleotidic sequence of RNA (mRNA) or of DNA (cDNA).

The aforesaid alternative splicing isoform according to the present invention, can be labelled or fused to an oligonucleotidic sequence encoding for a protein marker, preferably selected from the group consisting of luciferase, green fluorescent protein (GFP) and its variants as well as fluorescent proteins of various type, epitopes of various type c-myc, tag recognized by monoclonal or polyclonal antibodies, fluorescent dyes, biotin.

It is another object of the present invention the above defined alternative splicing isoform for the use in medical field.

Further object of the present invention is represented by the amino acidic sequence encoded by the alternative splicing isoform as above defined. Preferably, said amino acidic sequence includes the following amino acidic sequence:

(SEQ ID NO: 2)

MTFDDLKIQT VKDQPDEKSN GKKAKGLQFL YSPWWCLAAA
TLGVLCLGLV VTIMVLGMQL SQVSDLLTQE QANLTHQKKK
LEGQISARQQ AEEASQESEN ELKEMIETLA RKLNEKSKEQ
MELHHQNLNL QETLKRVANC SAPCPQDWIW HGENCYLFSS
GSFNWEKSQE KCLSLDAKLL KINSTADLF.

It constitutes further object of the present invention the aforesaid amino acidic sequence for the use in medical field.

Furthermore the present invention relates to the oligonucleotidic sequence or its complementary sequence encoding for the above mentioned amino acidic sequence.

The present invention further concerns a vector comprising the oligonucleotidic sequence of the alternative splicing isoform or the oligonucleotidic sequences encoding for the above defined protein. Additionally the present invention pertains to the above defined vector for the use in medical field.

It is another object of the present invention the use of the alternative splicing isoform or the amino acidic sequence or the vector as above defined, for the preparation of an antiapoptotic medicament that inhibits the instability of the atherosclerotic plaque.

It constitutes an object of the present invention the use of the alternative splicing isoform or the amino acidic sequence or the vector as above defined, as marker for the determination of the risk of cardiovascular diseases. Preferably, the prediction of the risk of cardiovascular diseases is effected estimating the quantitative ratio between the native oligonucleotidic or amino acidic LOX-1 isoform and the corresponding marker. More preferably, said cardiovascular diseases are selected from the group consisting of atherosclerosis, myocardial infarction, coronary atherosclerotic disease, cerebral stroke, ischemia, acute and chronic peripheral vascular diseases.

Furthermore, the present invention refers to a method for the determination of the risk of cardiovascular diseases, preferably selected from the group consisting of atherosclerosis, myocardial infarction, coronary atherosclerotic disease, cerebral stroke, ischemia, acute and chronic peripheral vascular diseases, comprising the following steps:
a) quantitative detection of m-RNA levels of native LOX-1 and the alternative splicing isoform according to the invention in a biological sample, preferably in the peripheral blood;
b) determination of the value of the ratio R LOX-1 transcript/alternative splicing isoform transcript as defined in claims 1 to 3 and of the relative risk according to the following values:

| | |
|---|---|
| Low risk | R < 0.6 |
| Intermediary risk | 0.7 < R < 0.8 |
| High risk | R > 0.9. |

Preferably, the detection of step a) is carried out by Real-Time PCR and at least one oligonucleotidic probe and at least one pair of specific primers for native LOX-1 and/or of the alternative splicing isoform mRNAs according to the present invention. According to a preferred embodiment of the method of the present invention the detection is carried out by two oligonucleotidic probes and two pairs of primers able to selectively quantify the two isoforms: LOX-1 and the alternative splicing isoform LOXIN, said probes comprising the following oligonucleotidic sequences:

| | |
|---|---|
| i) Probe LOX-1 | |
| 5′ - FAM-AGCTGATCTGGACTT-3′; | (SEQ ID NO: 3) |
| ii) Probe LOXIN | |
| 5′ - VIC-CAGCTGATCTGATTT-3′; | (SEQ ID NO: 4) |
| said pairs of primers comprising: | |
| iii) Primer LOX-1 Fw | |
| 5′ - TGCCAAGTTGCTGAAAATTAATAGC-3′; | (SEQ ID NO: 5) |
| vi) Primer LOX-1 Rv | |
| 5′ - AACTGGAATAGGAAATTGCTTGCT-3′; | (SEQ ID NO: 6) |
| v) Primer LOXIN Fw | |
| 5′ - TGCCAAGTTGCTGAAAATTAATAGC-3′; | (SEQ ID NO: 7) |
| vi) Primer LOXIN Rv | |
| 5′ - TGAAGGGTATGTCTGGGAGACA-3′. | (SEQ ID NO: 8) |

It is an object of the present invention a diagnostic kit comprising the oligonucleotidic probes and the specific primers for the native LOX-1 isoform and for the alternative splicing LOXIN isoform according to the invention for the prediction of the risk of cardiovascular diseases, wherein said probes comprise the following oligonucleotidic sequences:

| | |
|---|---|
| i) Probe LOX-1 | |
| 5′- FAM-AGCTGATCTGGACTT-3′; | (SEQ ID NO: 3) |
| ii) Probe LOXIN | |
| 5′- VIC-CAGCTGATCTGATTT-3′; | (SEQ ID NO: 4) |
| and | |

said pairs of primer comprise the following
oligonucleotidic sequences:

| | |
|---|---|
| iii) Primer LOX-1 Fw | |
| 5′- TGCCAAGTTGCTGAAAATTAATAGC-3′; | (SEQ ID NO: 5) |
| iv) Primer LOX-1 Rv | |
| 5′- AACTGGAATAGGAAATTGCTTGCT-3′; | (SEQ ID NO: 6) |
| v) Primer LOXIN Fw | |
| 5′- TGCCAAGTTGCTGAAAATTAATAGC-3′; | (SEQ ID NO: 7) |
| vi) Primer LOXIN Rv | |
| 5′- TGAAGGGTATGTCTGGGAGACA-3′. | (SEQ ID NO: 8) |

Preferably, said cardiovascular diseases are selected from the group consisting in atherosclerosis, myocardial infarction, coronary atherosclerotic disease, cerebral stroke, ischemia, acute and chronic peripheral vascular disease.

The present invention also refers to a probe for the detection of the alternative splicing isoform LOXIN according to the invention comprising the following nucleotidic sequence:

5′-CAGCTGATCTGATTT-3′ (SEQ ID NO:4) and to a probe for the detection of the native LOX-1 isoform comprising the following nucleotidic sequence:

5′-AGCTGATCTGGACTT-3′ (SEQ ID NO:3). Preferably, said probes are labelled with a substance selected from the group consisting of fluorophores and radioisotopes, and more preferably said fluorophore is 6-carboxyl fluorescein.

Further object of the present invention are the amplification primers of the alternative splicing isoform LOXIN as above defined comprising the following oligonucleotidic sequences:

| | |
|---|---|
| Fw | |
| 5′ - TGCCAAGTTGCTGAAAATTAATAGC-3′; | (SEQ ID NO: 7) |
| Rv | |
| 5′ - TGAAGGGTATGTCTGGGAGACA-3′. | (SEQ ID NO: 8) |

and the amplification primers of the native LOX-1 isoform comprising the following oligonucleotidic sequences:

| | |
|---|---|
| Fw | |
| 5′- TGCCAAGTTGCTGAAAATTAATAGC-3′; | (SEQ ID NO: 5) |
| Rv | |
| 5′- AACTGGAATAGGAAATTGCTTGCT-3′. | (SEQ ID NO: 6) |

The object of the present invention are also cells transfected with the vector according to the invention.

Finally, the present invention relates to a pharmaceutical composition comprising the alternative splicing isoform or the amino acidic sequence or the vector as above defined, as active principle along with one or more pharmaceutically acceptable adjuvants and/or excipients.

The present invention will be now described, for illustrative but not limitative purposes, according to its preferred embodiments, with particular reference to the figures of the enclosed drawings and examples, wherein:

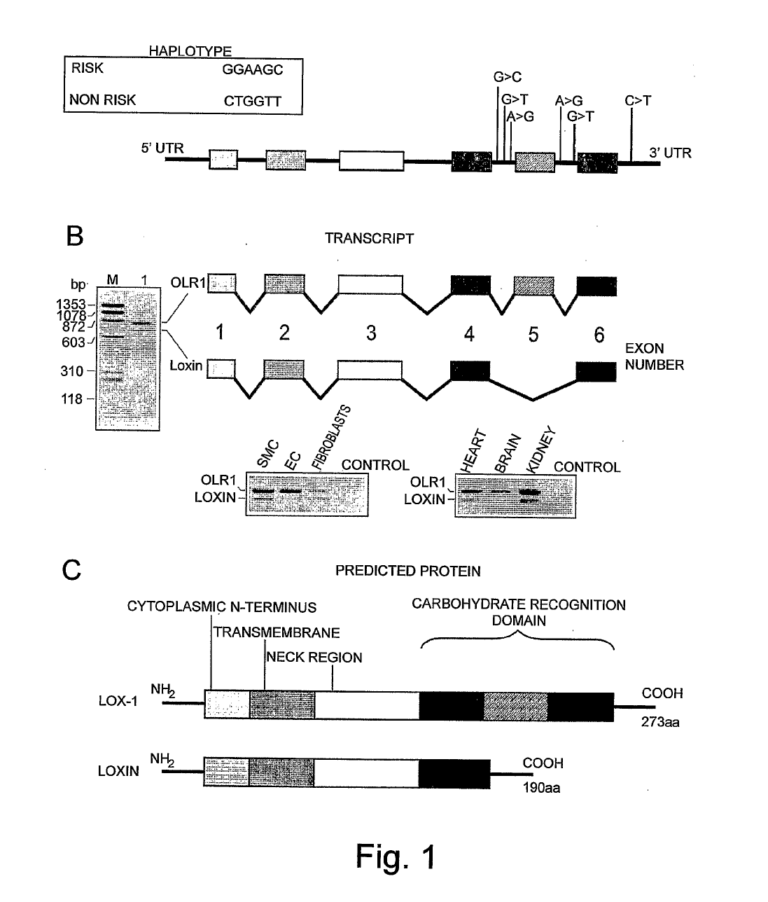

FIG. 1, panel A, shows genomic organization of the OLR1 gene whose exon-intron structure is shown along with SNPs position in the first part of 3′ non coding region, in complete linkage disequilibrum (LD); the side box represents the “risk” and “non-risk” haplotype at the LD block; panel B shows identification of alternatively spliced forms of OLR1 gene and expression pattern in different cell types and tissues as indicated; panel C shows a predicted protein schematic representation,

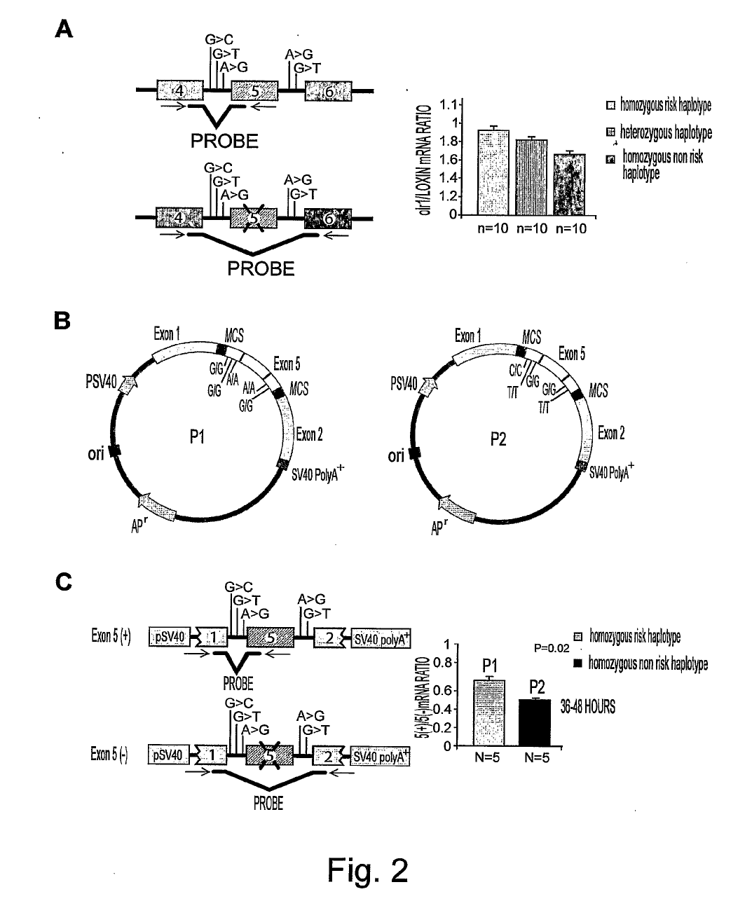

FIG. 2 shows how SNPs located in the LD block (linkage disequilibrum) modulate the levels of the OLR1 mRNA isoforms; panel A shows quantification of the OLR1 and LOXIN transcripts using quantitative real-time PCR; graphs show relative amounts of the two expressed isoform quantitative as ratio between OLR1 isoform (containing exon 5) and LOXIN isoform (lacking exon 5); panel B shows the map of the two minigene plasmids P1 and P2 containing the genomic sequence homozygous for the “risk” haplotype and the “non-risk” haplotype, respectively; panel C shows quantification of the P1 and P2 minigenes expression; graphs show the ratio between the transcript containing exon 5 and transcript lacking exon 5;

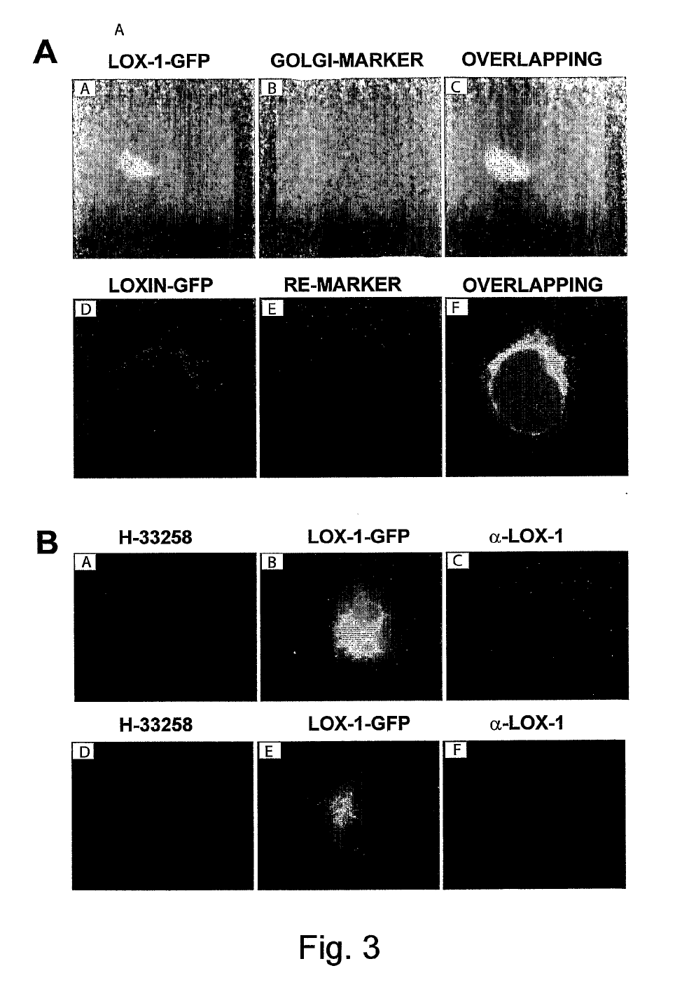

FIG. 3 shows subcellular distribution of LOX-1 and LOXIN isoforms; panel A and panel D show COS-7 cells transfected with LOX-1-GFP and LOXIN-GFP respectively; panel B and panel E show the same cells costained with antibodies against the Golgi 58K protein and calnexin; panels C and F represent the merged images; in panel B, unpermeabilised transfected COS-7 cells were immunostained with anti-LOX-1 antibodies;

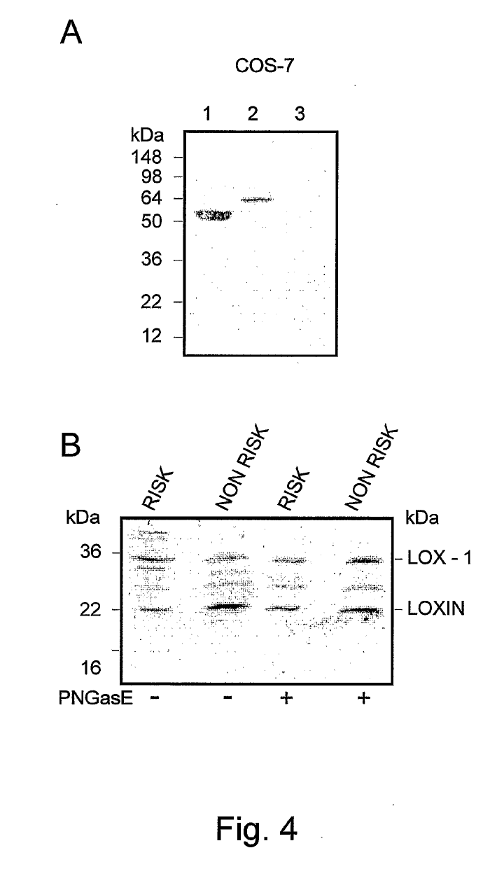

FIG. 4 shows intracellular expression of LOX-1 and LOXIN isoforms; panel A shows Western blot analysis of cellular lysates derived from mock-transfected (lane 3) or COS-7 cells transiently transfected with DNA encoding for LOXIN-GFP (lane 1) and LOX-1-GFP (lane 2); the two isoforms were detected with rabbit anti-LOX-1 antibodies; panel B shows Western blot analysis of cellular lysates of PBMCs derived from subjects carrying the “risk” (lanes 1 and 3) and “non-risk” (lanes 2 and 4) haplotypes incubated for 3 hours with PNGase F (1000 U), as indicated;

FIG. 5 shows the protective effect of LOXIN expression; panel A shows percentage of apoptotic cells in isolated monocytes-macrophages samples isolated from PBMCs of 10 subjects homozygous for the “risk” haplotype and 10 for the “non-risk” haplotype; panel B, shows COS-7 fibroblasts transiently transfected with the vector encoding for LOXIN-GFP and LOX-1-GFP alone or cotransfected with a fixed amount of LOX-1 and increasing concentration of LOXIN-GFP plasmids (as indicated); the average of the percentage of apoptotic cells in cells transfected with GFP alone (6%) was subtracted to all experimental points; the results shown represent average of three independent experiments.

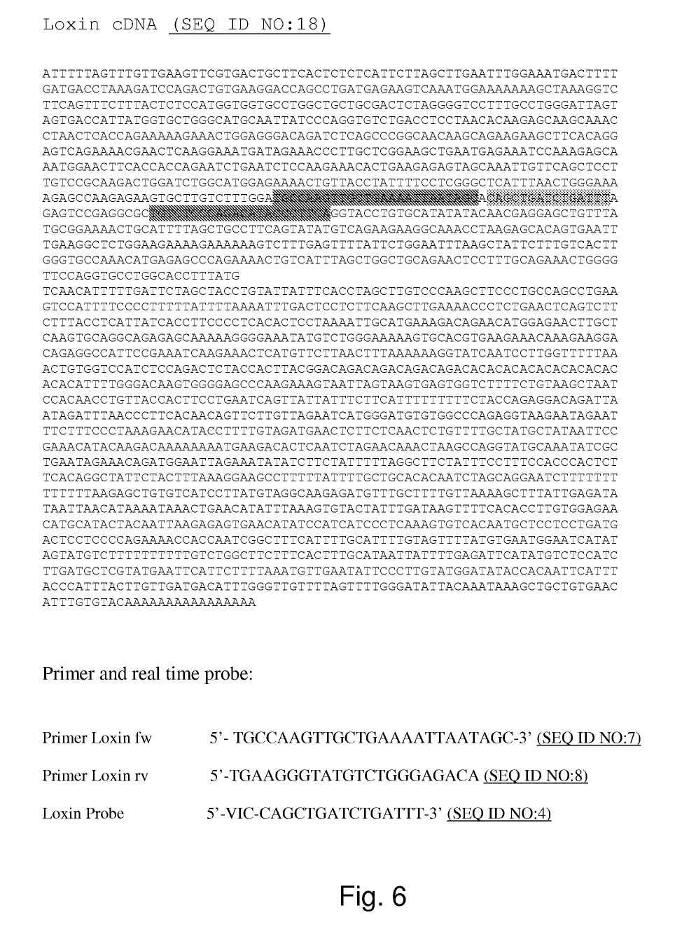

FIG. 6 shows the sequence of the LOXIN cDNA, Fw and Rv primers and the probe labelled with VIC fluorochrome thereof.

### EXAMPLE 1

### Identification of Alternative Isoforms of the OLR1 Gene

This study reports, firstly, the identification and characterization of a new functional isoform of the ORL1 gene and provides a functional characterization of the genetic association between SNPs within ORL1 gene and myocardial infarction.

It has been demonstrated that SNPs located in the linkage disequilibrium (LD) block modulate the relative abundance of the two transcripts: OLR1 (native transcript) and LOXIN (alternative splicing isoform). Both in vivo and in vitro experiments indicate that the new splice variant LOXIN is expressed at a similar level as the full-length receptor LOX-1. Macrophages from subjects carrying the “non-risk” haplotype express more LOXIN and result in fewer cells undergoing apoptosis on oxLDL induction. Macrophages play an important role in all phases of formation of atherosclerosis plaque, from the development of the fatty steak to the process that ultimately contribute to plaque rupture and myocardial infarction (Li AC, Glass CK. The macrophage foam cell as a target for therapeutic intervention. Nat Med. 8; 1235-1242; Kavurma M M, Bhindi R, Lowe H C, Chesterman C, Khachigian L M. Vessel wall apoptosis and atherosclerotic plaque instability. J Thromb Haemost. 3; 465-472; Tabas I). Apoptosis and plaque destabilization in atherosclerosis: the role of macrophage apoptosis induced by cholesterol. Cell Death Differ. 11; 12-16). Indeed, evidence from pathological studies link apoptosis of plaque resident macrophages with rupture and thrombosis of atherosclerotic lesions and subsequent development of acute vascular complication.

In particular, it has been proposed that extensive apoptosis of macrophages occurs only at sites of plaque rupture and possibly contributes to the process of rupture and thrombosis (Kolodgie F D, Narula J, Burke A P, Haider N, Farb A, Hui-Liang Y, Smialek J, Virmani R. Localization of apoptotic macrophages at the site of plaque rupture in sudden coronary death. Am J Pathol. 157; 1259-1268). It is our finding that increased levels of LOXIN and reduced levels of apoptosis in macrophages suggest that this in turn could result in plaque stabilization by influencing the nature of the vulnerable plaque. The study shows that different cellular localization of the two isoforms by mean of immunofluorescence studies. In cell lines transfected with the LOXIN-GFP construct we show that LOXIN isoform displays no surface expression in 90% of transfected cells. This is attributable to retention of LOXIN in the endoplasmic reticulum ER and accumulation of the protein in the perinuclear region. Regulation of cellular trafficking of LOX-1 receptor to the plasma membrane may provide an essential mechanism for the control of its function in vivo. In this context, different splice variant isoforms in the C-terminal domain have been reported for other receptors, such as glutamate receptors. In this case, the domain has been shown to be the site of interactions of proteins involved in the trafficking and stabilization of glutamate receptors in the synaptic membrane and, in turn, to play a role in synaptic strength and plasticity (Barry M F, Ziff E B. Receptor trafficking and the plasticity of excitatory synapses. Curr Opin Neurobiol. 12; 279-286; Carroll R C, Zukin R S. NMDA-receptor trafficking and targeting: implications for synaptic transmission and plasticity. Trends Neurosci. 25; 571-577).

Whether LOXIN forms heteromeric receptors with LOX-1 and regulates its intracellular traffic in vivo is under study.

LOX-1 receptor, when ectopically expressed in fibroblasts, is toxic and results in a high percentage of cells undergoing apoptosis. We demonstrate here that LOXIN molecules, when coexpressed with LOX-1 receptors, have a protective role and are able to rescue the apoptotic phenotype. The mechanism by which LOXIN exerts its protective role is not known but we propose different hypotheses. First, LOXIN may act on LOX-1 receptors by forming inactive heterodimers. Because the LOXIN splice variant lacks exon 5, it misses the binding region of ox-LDL. By increasing the intracellular LOXIN relative amount, the number of functional receptors able to bind oxLDL may be reduced. Because oxLDL induces apoptosis in macrophages, thereby reducing oxLDL binding sites, fewer cells will go through apoptosis. Secondly, LOXIN may exert its action on the transport of LOX-1 receptors toward the cellular membrane, blocking them into the ER and downregulating their membrane expression. Thirdly, the LOXIN itself may even have an antiapoptotic activity. These different hypotheses warrant further studies.

It is important to note that the expression level of both isoforms in vivo is similar. Subjects homozygous for “nonrisk” haplotype show a small increase in LOXIN expression compared with homozygous for “risk” haplotype. In vitro experiments confirmed this fine balance between the 2 isoforms. A small amount of LOXIN is sufficient to rescue the lethal phenotype induced by LOX-1 polymorphism localized in the LD block of OLR1 gene, with their functional role, may represent an important genetic risk factor for prediction of susceptibility to myocardial infarction. Moreover LOXIN could represent a new target for prevention of progression and destabilization of atheroslerotic plaque. Therefore, new research or therapeutic strategies favoring increasing expression of LOXIN could be effective for preventing plaque instability.

### Materials and Method

### RT-PCR Experiments

Total RNA was purified from monocyte-derived macrophages and COS-7 cells using RNeasy Mini Purification kit (Qiagen). Poly A⁺ RNA were purified using the Oligotex™ mRNA Mini Kit (Qiagen). Reverse transcription was performed with a High-Capacity cDNA Archive Kit (Applied Biosystems). RT products were amplified using forward OLR1-FW primer (5′-TGTTGAAGTTCGTGACTGCTT-3′ (SEQ ID NO:9)) and reverse OLR1-RW primer (5′-TTCTGCAGCCAGCTAAATGA-3′ (SEQ ID NO:10)), and then subcloned using a TA cloning kit (Invitrogen).

### DNA Constructs

Minigene: To amplify the genomic sequence surrounding the alternatively spliced exon 5 of OLR1 gene, we used the primer pair OLR1-XholF (5′-ACA GTC CTC GAG GTG AGT GTT CAT GGA TAT TTG-3′ (SEQ ID NO:11)) and OLR1-EcoRIR (5′TGT GTG GATATC CTG CAG GTA GGA AAA ACA AAA-3′ (SEQ ID NO:12)). We used human genomic DNA homozygous with respect to “risk” or “non-risk” haplotype at LD block of OLR1 gene as the PCR template. The PCR products were cloned into pSPL3 vector (Invitrogen) and sequenced using the primer OLR1SEQF (5′-GTT TCC TAT TCT TTG CTG AAC-3′ (SEQ ID NO:13)) and OLR1SEQR (5′-GTG GGG AGT AAT GTT TCT GAG-3′ (SEQ ID NO:14)). Recombinant protein: To generate LOX-1-GFP and LOXIN-GFP constructs, coding sequences of OLR1 gene and the splicing variant LOXIN were PCR-amplified from cDNA, which was derived from the human heart poly A⁺ RNA (Clontech) using selected oligos. For the amplification of OLR1, primers F1 (5′CCGCTCGAGATGACTTTTGATGACCTAAAG 3′ (SEQ ID NO:15)) and F2 (5′CGCGGATCCT GTGCTCTTAGGTTTGC 3′ (SEQ ID NO:16)) were used. For the amplification of LOXIN, primers F1 and F3 (5′CGCGGATCCATCA GATCAGCTGTGCTATT 3′ (SEQ ID NO:17)) were used. The PCR products were cloned into the Xhol/BamHI-digested pEGFP-N1 vector (Clontech) and sequenced using CEQ2000 (Beckman-Coultard).

### Quantitative PCR Real-Time

Real-time RT-PCR was performed on a TaqMAN ABI 7000 Sequence Detection System (Applied Biosystems). By using the Primer Express 2.0 software (Applied Biosystems), we designed primers and MGB probes for specific quantitative analysis of native transcript (OLR1) and the alternatively spliced isoforms. The sequences of the primers and of the MGB probes are the followings:

| | |
|---|---|
| i) Probe LOX-1 | |
| 5′ - FAM-AGCTGATCTGGACTT-3′; | (SEQ ID NO: 3) |
| ii) Probe LOXIN | |
| 5′ - VIC-CAGCTGATCTGATTT-3′; | (SEQ ID NO: 4 |

and said pairs of primer comprising the following oligonucleotide sequences:

| | |
|---|---|
| iii) Primer LOX-1 Fw | |
| 5′ - TGCCAAGTTGCTGAAAATTAATAGC-3′; | (SEQ ID NO: 5) |
| iv) Primer LOX-1 Rv | |
| 5′ - AACTGGAATAGGAAATTGCTTGCT-3′; | (SEQ ID NO: 6) |
| v) Primer LOXIN Fw | |
| 5′ - TGCCAAGTTGCTGAAAATTAATAGC-3′; | (SEQ ID NO: 7) |
| vi) Primer LOXIN Rv | |
| 5′ - TGAAGGGTATGTCTGGGAGACA-3′. | (SEQ ID NO: 8) |

Commercially available predeveloped TaqMan endogenous reference GAPDH gene (Applied Biosystems) was used to normalize the amount of cDNA added per sample. A comparative CT method was used to determine relative quantification of RNA expression. All PCR reactions were performed in triplicate.

Results are represented as a ratio between expression levels of native transcript (OLR1) normalized for the control gene (GAPDH) and the expression levels of alternative splicing isoform (LOXIN) normalized for the control gene (GAPDH).

### Cell Culture and Transfection

Human monocytes were isolated from peripheral blood mononuclear cells (PBMCs) of subjects homozygous for the “risk” and “non-risk” haplotype. We promoted their transition to macrophages in vitro as previously described, (Sawamura T, et al., Nature, 386; 73-77). Differentiation was determined by flow cytometry using anti-CD36 FITC monoclonal antibody (cell purity>95%). COS-7 fibroblasts were grown in Dulbecco's modified Eagle's medium (DMEM; Invitrogen) supplemented with 10% fetal bovine serum (FBS). Transient transfection of COS-7 was performed using the Nucleofector kit (Amexa Biosytems). 1 μg of plasmid DNA was added to 5×10⁵ COS-7 suspended in 100 μL of human dermal fibroblast Nucleofector solution (Amexa Biosytems). A-24 program was selected for a high density of transfection. All subjects gave informed consent, and the study protocol was approved by the Tor Vergata University Ethics Committee.

### Evaluation of Apoptosis

Differentiated macrophages were cultured without serum for 15 hours and then incubated with oxLDL (100 μg/mL; Intracel) for 8 hours before detection of apoptosis. Apoptosis was evaluated by multiparameter flow cytometry using a method that distinguishes nuclei from apoptotic, necrotic, or viable lymphoid cells (Matteucci C, et al., Cytometry. 35; 145-153). Isolated nuclei were analyzed by fluorescence and by forward- and side-angle scatter multiparameter analysis using a FACSscan Flowcytometer (Becton Dikinson). A minimum of 5000 events was collected for each sample. Apoptotic fibroblasts were visualized by staining with the blue fluorescent dye Hoechst 33342 (Sigma) and phosphatidylserine assay as described previously (Filesi I, et to the., Journal of Cell Science 115; 1803-1813). Annexin V and Hoechst 33342 positive cells were counted from cells transfected with GFP (green fluorescent protein), LOX-1-GFP, and LOXIN-GFP recombinant proteins.

### Immunofluorescence Staining

Immunofluorescence was performed as described. (Heinloth A, et al., Atherosclerosis. 162; 93-101). Affinity purified anti-rabbit LOX-1 (Santa Cruz), goat anti-calnexin (Santa Cruz), and mouse anti-Golgi 58K (Sigma) protein were used as primary antibodies. Texas Red goat anti-rabbit IgG (Calbiochem), Texas Red goat anti-mouse IgG (Calbiochem), and Rhodamine Red-Xconjugated affinipure rabbit anti-goat IgG (Jackson Immunoreasearch) were used as secondary antibodies. Hoechst 33342 dye was used at 1 μg/mL. Samples were examined with a DMRA Leica fluorescence microscope equipped with CCD camera. Acquired images were deconvolved using Leica Q-fluoro software and processed using Adobe Photoshop.

### Western Blot Analysis and Enzymatic Digestion

PBMCs and COS-7 cells were lysed for 30 minutes in ice-cold cell extraction buffer (EB) (100 mmol/L NaCl, 10 mmol/L EDTA, 0.5% Nonidet P-40, 0.5% sodium deoxycholate, 10 mmol/L Tris-HCl, pH 7.4, 1 mmol/L PMSF, 10 μg/mL pepstatin A, 10 μg/mL leupeptin and 0.3 μM/L aprotinin). Nuclei and large debris were removed by centrifugation at 290 g for 10 minutes at 4° C. The supernatants were then precipitated with 5 volumes of MeOH at −20° C. for 2 hours. After centrifugation (16000 g, 10 minutes, 4° C.), protein pellets were dissolved in 4 sample buffer (500 mmol/L Tris-HCl, pH 6.8, 4% SDS, 20% glycerol, 40 mmol/L DTT and 0.02% bromophenol blue) and heated at 95° C. for 5 minutes.

Blots were probed with rabbit anti-LOX-1 antibody (Santa Cruz).

Immunoreactive bands were detected with sheep anti-rabbit IgG horseradish peroxidase (Amersham) and visualized by ECL (Sigma). Enzymatic deglycosylation was performed as previously described.(Cardinal To, et to the., Methods. 34; 171-178).

### Statistic Analysis

All statistical analyses were performed using SPSS version 13 software. Data are presented as means ±1 standard deviation (SD). Normal distribution of continuous variables has been verified by Kolmogorov-Smirnov with Lillefors correction. Differences in means of continuous variables were analyzed by impaired t test or ANOVA as needed. Bonferroni correction was used for multiple comparison.

Results

Identification of an Alternatively Spliced Forms of OLR1 Gene

We started from total or poly(A)+ RNA of human monocytes derived macrophages and subjected it to reverse transcription to test the potential effect of the SNPs located in the LD block on RNA splicing of the human OLR1 gene. We performed PCR amplification using primers located in the 5′UTR and 3′UTR of the OLR1 gene and sequenced the amplified fragments. This analysis identified a reproducible pattern of alternative splicing around exon 5 (from nucleotide 565 to nucleotide 680 of the sequence of OLR transcript). In particular we identified two OLR1 transcripts in both RNA fractions. One of these products corresponded to the full-length transcript (OLR1), while the other lacked exon 5; we named it LOXIN (FIG. 1B). The newly spliced mRNA has a stop codon in the open reading frame that leads to a premature termination of the translation product and generates a predicted protein that lacks ⅔ of the lectin-like domain (FIG. 1C). Both isoforms were detected in several cell types (endothelial cells, fibroblasts, and smooth muscle cells), and tissues (heart, kidney, and brain), suggesting that they reflect a physiological pattern of expression of the OLR1 gene (FIG. 1B)

SNPs Located in the LD Block Modulate the Levels of mRNA Isoforms

To find in vivo evidence that SNPs located in the LD block modulate the level of the two mRNA isoforms, we performed an isoform-specific real-time PCR starting from total RNA of human monocyte-derived macrophages of selected patients carrying the “risk” and “non-risk” haplotype at OLR1 gene. By this analysis we noted a marked difference in the mRNA ratio (OLR1/LOXIN) according to the haplotype. In particular, the OLR1/LOXIN mRNA ratio was 33% higher in human monocyte-derived macrophages of subjects homozygous for the “risk” haplotype compared with homozygous for the “non-risk” haplotype (FIG. 2A).

The ratio R of OLR1/LOXIN mRNAs has been correlated to the risk of myocardial infarction:

| | |
|---|---|
| Low risk | R < 0.6 |
| Intermediate risk | R 0.7-0.8 |
| High risk | R > 0.9. |

The finding that the relative amount of the LOXIN transcript is significantly greater in subjects carrying the “non-risk” haplotype strongly suggests a negative link between levels of LOXIN mRNA and the incidence of myocardial infarction in humans.

To further confirm the regulatory role of the intronic polymorphism, we extended these studies by constructing minigenes carrying the “risk” and “non-risk” haplotypes with genomic sequences containing intron 4, exon 5, and intron 5 (FIG. 2B). These constructs were transfected in COS-7 fibroblasts and the ratio of unspliced (exon 5+) to spliced (exon 5−) transcript was analyzed by real time isoform specific PCR. As can be seen in FIG. 2C, the ratio of the unspliced form (5+) vs the spliced form (5−) was 27% higher in RNA extracted from cells transfected with minigenes carrying the “risk” haplotype. These in vitro experiments not only suggest that the relative abundance of the two isoforms is modulated by the intronic SNPs mapping within the LD block, but also confirm the previously described in vivo results (FIG. 2A).

### Subcellular and Membrane Distribution of LOX-1 and the LOXIN Splice Variant

To investigate the cellular localization of the natiuve transcript (OLR1) and the splice variant, we constructed two plasmids that allowed the efficient expression of LOX-1 and LOXIN in mammalian cells fused, C terminally, to the green fluorescent protein (GFP). Immunofluorescence analysis of transfected COS-7 fibroblasts revealed that the intracellular expression of the full-length LOX-1-GFP causes a cell-lethal phenotype. Many transfected cells are roundly shaped and tend to detach from the dish. On the contrary, LOXIN-GFP isoform is efficiently expressed, and its expression does not result in cytotoxicity. Notwithstanding the toxic effect, many LOX-1-GFP transfected cells retain normal morphology, and this has allowed us to study its intracellular distribution.

As shown in FIG. 4A, (panels A-C), LOX-1 distributed in the ER and in the Golgi apparatus. At 24 hours after transfection, LOX-1 colocalized almost exclusively with the Golgi 58K protein, indicating that LOX-1 receptor in the initial phase of synthesis traffics along the secretory pathway. In contrast, LOXIN-GFP was not detectable in the Golgi patches of permeabilized COS-7 cells. In these cells we observed a more widespread staining, characteristic of typical ER distribution. In 40% to 50% of cells transfected with LOXIN-GFP, an accumulation of fluorescence in the perinuclear area was also detected (FIG. 4A, panels D-F). The two GFP-tagged isoforms were also labeled for surface receptors in live cells (FIGS. 3B and 4). At 24 hours after transfection, over 90% of cells expressing LOX-1-GFP showed a typical punctuate plasma membrane-associated fluorescence (FIG. 4B, panel C). Remarkably, less than 10% of COS-7 cells transfected with LOXIN-GFP showed surface staining (FIG. 3B, panel F). The very low expression of LOXIN at the plasma membrane demonstrates that truncation of the C-terminal portion in LOXIN protein leads to a profound effect on cellular trafficking of the protein to the plasma membrane.

The expression level of the two isoforms in transfected fibroblasts was also confirmed in Western blot. A single band corresponding to LOX-1-GFP and LOXIN-GFP fusion proteins were detected (FIG. 4A, lanes 1 and 2). The molecular weight of the bands corresponded to the predicted molecular weight of nonglycosylated proteins. The LOX-1-GFP band was, however, much fainter, probably because of the previously mentioned cytotoxic effect of this construct (FIG. 4A, lane 2).

### In Vivo Expression of LOX-1 and LOXIN Isoforms

To verify whether LOXIN transcript is indeed translated in vivo and to analyze the level of its expression, we used Western blot to examine the relative amounts of the two isoforms in PBMCs derived from selected subjects with different haplotypes. As shown in FIG. 4B, immunoreaction of cell lysates showed 2 major bands of 34 and 22 kDa, corresponding to the predicted molecular weight of the two isoforms. Interestingly, we noticed a relative increase in the amount of LOXIN in cells derived from subjects homozygous for the “non-risk” haplotype (FIG. 4B, lanes 2 and 4). Removal of N-linked glycans by PNGase digestion (FIG. 4B, lanes 3 and 4) resulted in the disappearance of few faint bands, indicating that the two bands, 34 and 22 kDa, correspond to the unglycosylated LOX-1 and LOXIN proteins. In many gels, including the one shown in FIG. 4B, a third band of 26 kDa was also observed. This band may represent a degradation product of the LOX-1 protein that we are currently studying.

### In Vivo Proapoptotic Effect of LOX-1 and Rescue by LOXIN

We considered that an altered balance between the 2 isoforms could be related to the increased susceptibility to apoptosis.

To test this notion, we analyzed oxLDL-induced apoptosis in a monocytes/macrophages in vivo assay (Heinloth A, et al., Atherosclerosi, 162; 93-101). Human monocytes were isolated from buffy coats of healthy donors carrying the “risk” and “non-risk” haplotype at LD block of OLR1 gene. After Ficoll gradient centrifugation, monocytes were separated to induce differentiation in macrophages. 100 μg/mL of oxLDL was added to these cells to induce apoptosis. Remarkably, flow cytometric analysis of macrophages derived from subjects homozygous for the “non-risk” haplotype showed a 24% reduction in apoptotic cell number compared with the homozygous for the “risk” haplotype when treated with the oxLDL (FIG. 5A). Our data suggest that increased levels of LOXIN in macrophages may relate to reduced level of apoptosis.

Because the two isoforms are physiologically coexpressed in PBMCs with a different balance related to the haplotype, and increased LOXIN levels relate to the “non-risk” haplotype, we explored the possibility that LOXIN isoform may have a protective effect versus apoptosis. To test this hypothesis, we
transfected COS-7 fibroblasts with DNA encoding for LOXIN-GFP and LOX-1-GFP alone or cotransfected with a fixed amount of LOX-1 and increasing concentration of LOXIN-GFP plasmids (as indicated in FIG. 5B). The phenotype of transfected cells was analyzed using 2 markers of apoptosis. First, we used Annexin V, which detects alteration at the level of the plasma membrane. Next, we examined the uptake of blue-fluorescent Hoechst 33342 dye, which stains the condensed chromatin of apoptotic cells more brightly than the chromatin of nonapoptotic cells. On the basis of the combined staining patterns of these dyes, we were able to distinguish between normal, apoptotic, and dead cells.

As above mentioned, cells expressing the LOX-1-GFP fusion protein did not thrive, with many of the cells exhibiting cell shrinkage, which is a feature of apoptosis. Thus, LOX-1-GFP alone resulted in 36% of cells developing apoptosis. In contrast, LOXIN expression was not toxic and the number of apoptotic cells was comparable to the mock-transfected cells.

Interestingly, the coexpression of LOXIN-GFP resulted in a complete dose-dependent rescue of the LOX-1-GFP-induced phenotype. It is worth noting that a very low dose of LOXIN, corresponding to a ratio of 1:4 with LOX-1, resulted in a 72% reduction in the number of apoptotic cells, and a 1:1 ratio of the 2 plasmids used for transfection completely prevented the phenotype. This finding suggests that small differences in LOX-1/LOXIN balance and, especially, a small increase in LOXIN expression, may have a very profound effect on the cytotoxic phenotype also in vivo.

The present invention has been described to illustrative title, but not limitative, according to his/her preferred forms of realization, but it is to intend him that variations e/o changes you/they can be introduced by the experts in the branch without for this to go out of the relative circle of protection, as defined by the attached claims.

1-21. (canceled)

22. A method for inhibiting atherosclerotic plaque stability comprising: administering to a subject in need thereof an effective amount of alternative splicing isoform of the OLR1 gene encoding for the oxidized low density lipoproteines receptor-1 (LOX-1), characterized in that exon 5 from the nucleotide 565 to the nucleotide 680 is excised in comparison to the corresponding native oligonucleotidic sequence of the human OLR1 transcript (NM 002543) or its complementary sequence, or of the vector comprising the oligonucleotidic sequence of said alternative splicing isoform or of the amino acidic sequence encoded thereby.

23. A method for determining the risk of cardiovascular disease in a subject, comprising measuring in a sample the amount of an alternative splicing isoform of the OLR1 gene encoding for the oxidized low density lipoproteines receptor-1 (LOX-1), characterized in that exon 5 from the nucleotide 565 to the nucleotide 680 is excised in comparison to the corresponding native oligonucleotidic sequence of the human OLR1 transcript (NM 002543) or its complementary sequence, or of the vector comprising the oligonucleotidic sequence of said alternative splicing isoform or of the amino acidic sequence encoded thereby,

24. The method according to claim 23, wherein said cardiovascular diseases are selected from the group consisting in atherosclerosis, myocardial infarction, atherosclerotic coronary disease, cerebral stroke, ischemia, acute and chronic peripheral vascular diseases.

25. The method according to claim 22, wherein said native oligonucleotidic sequence comprises the following oligonucleotidic sequence:
(SEQ ID NO: 1)
atgacttttg atgacctaaa gatccagact gtgaaggacc
agcctgatga gaagtcaaat ggaaaaaaag ctaaaggtct
tcagtttctt tactctccat ggtggtgcct ggctgctgcg
actctagggg tcctttgcct gggattagta gtgaccatta
tggtgctggg catgcaatta tcccaggtgt ctgacctcct
aacacaagag caagcaaacc taactcacca gaaaaagaaa
ctggagggac agatctcagc ccggcaacaa gcagaagaag
cttcacagga gtcagaaaac gaactcaagg aaatgataga
aacccttgct cggaagctga atgagaaatc caaagagcaa
atggaacttc accaccagaa tctgaatctc caagaaacac
tgaagagagt agcaaattgt tcagctcctt gtccgcaaga
ctggatctgg catggagaaa actgttacct attttcctcg
ggctcattta actgggaaaa cagccaagag aagtgcttgt
ctttggatgc caagttgctg aaaattaata gcacagctga
tctggacttc atccagcaag caatttccta ttccagtttt
ccattctgga tggggctgtc tcggaggaac cccagctacc
catggctctg ggaggacggt tctcctttga tgccccactt
atttagagtc cgaggcgctg tctcccagac atacccttca
ggtacctgtg catatataca acgaggagct gtttatgcgg
aaaactgcat tttagctgcc ttcagtatat gtcagaagaa
ggcaaaccta agagcacagt ga;
or its complementary sequence.

26. The method according to claim 22, wherein the oligonucleotidic sequence is RNA or DNA.

27. The method according to claim 26, said isoform being labelled or being fused with a oligonucleotidic sequence encoding for a protein marker.

28. The method according to claim 27, wherein said protein is selected from the group consisting of luciferase, green fluorescent protein (GFP) and its variants, fluorescent proteins, c-myc or tag epitopes recognized by monoclonal or polyclonal antibodies, fluorescent dyes, biotin.

29. The method according to claim 22, wherein said amino acidic sequence comprises the following amino acidic sequence:
(SEQ ID NO: 2)
MTFDDLKIQT VKDQPDEKSN GKKAKGLQFL YSPWWCLAAA
TLGVLCLGLV VTIMVLGMQL SQVSDLLTQE QANLTHQKKK
LEGQISARQQ AEEASQESEN ELKEMIETLA RKLNEKSKEQ
MELHHQNLNL QETLKRVANC SAPCPQDWIW HGENCYLFSS
GSFNWEKSQE KCLSLDAKLL KINSTADLF.

30. The method according to claim 22, wherein the oligonucleotidic sequence of the alternative splicing isoform encodes for the amino acidic sequence: of SEQ ID NO:2.

31. Method for the determination of the risk of cardiovascular diseases comprising the following steps:
a) quantitative detection of mRNAs levels of LOX-1 and the alternative splicing isoform as defined according to claim 23 in a biological sample;
b) determination of the value of the ratio R LOX-1 native transcript/alternative splicing isoform transcript as previously defined and of the relative risk on the following values basis:

| | |
|---|---|
| Low risk | R < 0.6 |
| Intermediate risk | R among 0.7-0.8 |
| High risk | R > 0.9. |

32. Method according to claim 31, wherein the detection of step a) is carried out by means of Real-Time PCR through at least one oligonucleotidic probe and at least a pair of specific primer specific for mRNAs of native LOX-1 and/or the alternative splicing isoform.

33. Method according to claim 32, wherein the detection is carried out by through two oligonucleotidic probes and two pairs of primer able to selectively quantify the two isoforms LOX-1 and the alternative splicing LOXIN isoforms, said probes comprise the following oligonucleotidic sequences:

| | |
|---|---|
| i) Probe LOX-1 | |
| 5′- FAM-AGCTGATCTGGACTT-3′; | (SEQ ID NO: 3) |

ii) Probe LOXIN 5′-VIC-CAGCTGATCTGATTT-3′ (SEQ ID NO:4); said pairs of primer comprising:

| | |
|---|---|
| iii) Primer LOX-1 Fw | |
| 5′- TGCCAAGTTGCTGAAAATTAATAGC-3′; | (SEQ ID NO: 5) |
| iv) Primer LOX-1 Rv | |
| 5′- AACTGGAATAGGAAATTGCTTGCT-3′; | (SEQ ID NO: 6) |
| v) Primer LOXIN Fw | |
| 5′- TGCCAAGTTGCTGAAAATTAATAGC-3′; | (SEQ ID NO: 7) |
| vi) Primer LOXIN Rv | |
| 5′- TGAAGGGTATGTCTGGGAGACA-3′. | (SEQ ID NO: 8) |

34. Method according to claim 31, wherein said cardiovascular diseases are selected from the group consisting in atherosclerosis, myocardial infarction, atherosclerotic coronary disease, cerebral stroke, ischemia, acute and chronic peripheral vascular diseases.

35. Method according to claim 31, wherein said biological sample is peripheral blood.

36. Diagnostic kit comprising oligonucleotidic probes and specific primers for the native LOX-1 isoform and for alternative splicing isoform LOXIN as defined according to claim 23, for the prediction of the risk of cardiovascular disease, in which said probes comprise the following oligonucleotidic sequences:

| | |
|---|---|
| i) Probe LOX-1 | |
| 5′- FAM-AGCTGATCTGGACTT-3′; | (SEQ ID NO: 3) |
| ii) Probe LOXIN | |
| 5′- VIC-CAGCTGATCTGATTT-3′; | (SEQ ID NO: 4) |

said pairs of primer comprise the following oligonucleotidic sequences:

| | |
|---|---|
| iii) Primer LOX-1 Fw | |
| 5′- TGCCAAGTTGCTGAAAATTAATAGC-3′; | (SEQ ID NO: 5) |
| iv) Primer LOX-1 Rv | |
| 5′- AACTGGAATAGGAAATTGCTTGCT-3′; | (SEQ ID NO: 6) |
| v) Primer LOXIN Fw | |
| 5′- TGCCAAGTTGCTGAAAATTAATAGC-3′; | (SEQ ID NO: 7) |
| vi) Primer LOXIN Rv | |
| 5′- TGAAGGGTATGTCTGGGAGACA-3′. | (SEQ ID NO: 8) |

37. Kit according to claim 36, wherein said cardiovascular diseases are selected from the group consisting in atherosclerosis, myocardial infarction, atherosclerotic coronary disease, cerebral stroke, ischemia, acute and chronic peripheral vascular diseases.

38. Probe for the detection of the alternative splicing isoform LOXIN as defined in claim 23 comprising the following nucleotidic sequence:

| | |
|---|---|
| 5′- CAGCTGATCTGATTT-3′. | (SEQ ID NO: 4) |

39. Probe according to claim 36, wherein said probe is labelled with a substance selected from the group consisting in fluorophore, radioisotope, luminescent substance.

40. Pharmaceutical composition comprising the alternative splicing isoform as defined according to claim 22, or the vector comprising the oligonucleotidic sequence of said alternative splicing isoform or the amino acidic sequence encoded thereby, as active principle along with one or more pharmacologically acceptable adjuvants and/or excipients.

41. Pharmaceutical composition according to claim 40, wherein said amino acidic sequence comprises the following amino acidic sequence:
(SEQ ID NO: 2)
MTFDDLKIQT VKDQPDEKSN GKKAKGLQFL YSPWWCLAAA
TLGVLCLGLV VTIMVLGMQL SQVSDLLTQE QANLTHQKKK
LEGQISARQQ AEEASQESEN ELKEMIETLA RKLNEKSKEQ
MELHHQNLNL QETLKRVANC SAPCPQDWIW HGENCYLFSS
GSFNWEKSQE KCLSLDAKLL KINSTADLF.

42. Pharmaceutical composition according to claim 39, wherein the oligonucleotidic sequence of the alternative splicing isoform encodes for the amino acidic sequence according to SEQ ID NO:2.


SEQUENCE LISTING (18 sequences)

SEQ ID NO: 1 - DNA, 822 bp, Artificial
  Alternative splicing isoform of OLR-1 gene
atgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcagctccttgtccgcaagactggatctggcatggagaaaactgttacctattttcctcgggctcatttaactgggaaaacagccaagagaagtgcttgtctttggatgccaagttgctgaaaattaatagcacagctgatctggacttcatccagcaagcaatttcctattccagttttccattctggatggggctgtctcggaggaaccccagctacccatggctctgggaggacggttctcctttgatgccccacttatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtga

SEQ ID NO: 2 - protein, 189 aa, Artificial
  Alternative splicing isoform aminoacid sequence
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLF

SEQ ID NO: 3 - DNA, 15 bp, Artificial
  LOX-1 probe
agctgatctggactt

SEQ ID NO: 4 - DNA, 15 bp, Artificial
  Loxin probe
cagctgatctgattt

SEQ ID NO: 5 - DNA, 25 bp, Artificial
  Fw LOX-1 primer
tgccaagttgctgaaaattaatagc

SEQ ID NO: 6 - DNA, 24 bp, Artificial
  Rv LOX-1 primer
aactggaataggaaattgcttgct

SEQ ID NO: 7 - DNA, 25 bp, Artificial
  Fw Loxin primer
tgccaagttgctgaaaattaatagc

SEQ ID NO: 8 - DNA, 22 bp, Artificial
  Rv Loxin primer
tgaagggtatgtctgggagaca

SEQ ID NO: 9 - DNA, 21 bp, Artificial
  OLR-1 Fw primer
tgttgaagttcgtgactgctt

SEQ ID NO: 10 - DNA, 20 bp, Artificial
  OLR-1 Rv primer
ttctgcagccagctaaatga

SEQ ID NO: 11 - DNA, 33 bp, Artificial
  OLR1-XhoIF primer
acagtcctcgaggtgagtgttcatggatatttg

SEQ ID NO: 12 - DNA, 33 bp, Artificial
  OLR-1-EcoRIR primer
tgtgtggatatcctgcaggtaggaaaaacaaaa

SEQ ID NO: 13 - DNA, 21 bp, Artificial
  OLR1SEQF sequencing forward primer
gtttcctattctttgctgaac

SEQ ID NO: 14 - DNA, 21 bp, Artificial
  OLR1SEQR sequencing reverse primer
gtggggagtaatgtttctgag

SEQ ID NO: 15 - DNA, 30 bp, Artificial
  OLR-1 amplification primer
ccgctcgagatgacttttgatgacctaaag

SEQ ID NO: 16 - DNA, 26 bp, Artificial
  OLR-1 amplification primer
cgcggatcctgtgctcttaggtttgc

SEQ ID NO: 17 - DNA, 29 bp, Artificial
  Loxin amplification primer
cgcggatccatcagatcagctgtgctatt

SEQ ID NO: 18 - DNA, 2360 bp, Artificial
  Loxin cDNA
atttttagtttgttgaagttcgtgactgcttcactctctcattcttagcttgaatttggaaatgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcagctccttgtccgcaagactggatctggcatggagaaaactgttacctattttcctcgggctcatttaactgggaaaagagccaagagaagtgcttgtctttggatgccaagttgctgaaaattaatagcacagctgatctgatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtgaatttgaaggctctggaagaaaagaaaaaagtctttgagttttattctggaatttaagctattctttgtcacttgggtgccaaacatgagagcccagaaaactgtcatttagctggctgcagaactcctttgcagaaactggggttccaggtgcctggcacctttatgtcaacatttttgattctagctacctgtattatttcacctagcttgtcccaagcttccctgccagcctgaagtccattttcccctttttattttaaaatttgactcctcttcaagcttgaaaaccctctgaactcagtcttctttacctcattatcaccttcccctcacactcctaaaattgcatgaaagacagaacatggagaacttgctcaagtgcaggcagagagcaaaaaggggaaatatgtctgggaaaaagtgcacgtgaagaaacaaagaaggacagaggccattccgaaatcaagaaactcatgttcttaactttaaaaaaggtatcaatccttggtttttaaactgtggtccatctccagactctaccacttacggacagacagacagacagacacacacacacacacacacacacattttgggacaagtggggagcccaagaaagtaattagtaagtgagtggtcttttctgtaagctaatccacaacctgttaccacttcctgaatcagttattatttcttcatttttttttctaccagaggacagattaatagatttaacccttcacaacagttcttgttagaatcatgggatgtgtggcccagaggtaagaatagaatttctttccctaaagaacataccttttgtagatgaactcttctcaactctgttttgctatgctataattccgaaacatacaagacaaaaaaaatgaagacactcaatctagaacaaactaagccaggtatgcaaatatcgctgaatagaaacagatggaattagaaatatatcttctatttttaggcttctatttcctttccacccactcttcacaggctattctactttaaaggaagcctttttattttgctgcacacaatctagcaggaatctttttttttttttaagagctgtgtcatccttatgtaggcaagagatgtttgcttttgttaaaagctttattgagatataattaacataaaataaactgaacatatttaaagtgtactatttgataagttttcacaccttgtggagaacatgcatactacaattaagagagtgaacatatccatcatccctcaaagtgtcacaatgctcctcctgatgactcctccccagaaaaccaccaatcggctttcattttgcattttgtagttttatgtgaatggaatcatatagtatgtcttttttttttgtctggcttctttcactttgcataattattttgagattcatatgtctccatcttgatgctcgtatgaattcattcttttaaatgttgaatattcccttgtatggatataccacaattcatttacccatttacttgttgatgacatttgggttgttttagttttgggatattacaaataaagctgctgtgaacatttgtgtacaaaaaaaaaaaaaaaa